(19) United States
(12) Patent Application Publication
(10) Pub. No.: US 2023/0372578 A1
(43) Pub. Date: Nov. 23, 2023

(54) Wound Dressing Material and Methods of Making and Using the Same

(71) Applicant: 3M INNOVATIVE PROPERTIES COMPANY, St. Paul, MN (US)

(72) Inventors: Petra L. Kohler Riedi, Minneapolis, MN (US); Saurabh Batra, Minneapolis, MN (US); Rajan B. Bodkhe, Woodbury, MN (US); Joseph J. Stoffel, Hastings, MN (US); Naimul Karim, Maplewood, MN (US)

(21) Appl. No.: 18/044,692

(22) PCT Filed: Aug. 10, 2021
(86) PCT No.: PCT/IB2021/057385

§ 371 Date: Mar. 9, 2023

Related U.S. Application Data
(60) Provisional application No. 63/082,249, filed on Sep. 23, 2020.

(51) Int. Cl. A61L 26/00; A61F 13/02; A61F 13/00

(52) U.S. Cl. CPC A61L 26/0052; A61L 26/0066; A61L 26/0085; A61F 13/0206; A61F 13/00063; A61L 2300/404; A61L 2300/21

(57) ABSTRACT

A wound dressing material comprises: a first water-sensitive film comprising a first copolymer comprising first divalent hydroxyethylene monomer units and first divalent dihydroxybutylene monomer units; and a first antimicrobial layer disposed on the first water-sensitive film. Methods of making and using the wound dressing material are also disclosed.

### TECHNICAL FIELD

The present disclosure broadly relates to antimicrobial wound dressing materials, to processes suitable for the preparation of such materials, and to the use of such materials as wound dressings.

### BACKGROUND

Traditionally, wet-to-dry gauze has been used to dress wounds. Dressings that create and maintain a moist environment, however, are now typically considered to provide optimal conditions for wound healing. Indeed, highly hydrophilic and absorbent wound dressing materials are part of the rapidly growing advanced wound care market. High-gelling fiber wound dressing products are popular with clinicians and are made of materials which absorb and hold moisture to create a gel-like environment to maintain moisture at the wound site. The most common materials used in these products are alginate and carboxymethyl cellulose.

Many wound care products include cationic antiseptics, which kill a wide variety of microorganisms, but are sequestered and/or deactivated by anionic materials such as alginate and carboxymethyl cellulose in the wound care product itself. Rayon is another highly hydrophilic material often used in wound care products, but it likewise also binds cationic antimicrobial molecules.

There is a continuing need for materials and articles to facilitate wound healing.

### SUMMARY

Advantageously, the present disclosure provides antiseptic wound dressing materials that provide a moist environment while providing antimicrobial protection, even in the presence of cationic antiseptics.

In one aspect, the present disclosure provides a first water-sensitive film comprising a first copolymer comprising first divalent hydroxyethylene monomer units and first divalent dihydroxybutylene monomer units; and
a first antimicrobial layer disposed on the first water-sensitive film.

In some embodiments, the wound dressing material further comprises a second water-sensitive film comprising a second copolymer comprising second divalent hydroxyethylene monomer units and second divalent dihydroxybutylene monomer units, wherein the second water-sensitive film is disposed on the first antimicrobial layer opposite the first water-sensitive film.

In some embodiments, the wound dressing material further comprises a porous core layer having first and second opposed major surfaces, wherein the first major surface of the porous core layer contacts the first antimicrobial layer. In some of these embodiments, the wound dressing material further comprises a flexible adhesive barrier film adhered to and proximate to the second major surface of the porous core layer. In some other embodiments the wound dressing material further comprises: a second antimicrobial layer disposed on the second major surface of the porous core layer; and a second water-sensitive film disposed on the second antimicrobial layer opposite the porous core layer, wherein the second water-sensitive film comprises a second copolymer comprising divalent hydroxyethylene monomer units and divalent dihydroxybutylene monomer units.

In another aspect, the present disclosure provides a method of using a wound dressing material according to the present disclosure, the method comprising contacting the first water-sensitive film with an exposed surface of a wound.

In another aspect, the present disclosure provides a method of making a wound dressing material according to the present disclosure, the method comprising laminating sequential layers:
a) a first water-sensitive film comprising a first copolymer comprising first divalent hydroxyethylene monomer units and first divalent dihydroxybutylene monomer units;
b) a first antimicrobial layer; and optionally
c) a porous core layer.

As used herein:
the term “film” refers to a thin continuous, non-fibrous layer of material; and
the term “water-sensitive” means water-soluble, water-dispersible, and/or water-degradable.

Features and advantages of the present disclosure will be further understood upon consideration of the detailed description as well as the appended claims.

### BRIEF DESCRIPTION OF THE DRAWINGS

FIG. 1 is a schematic side view of an exemplary wound dressing material 100 according to the present disclosure.

FIG. 2 is a schematic side view of an exemplary wound dressing material 200 according to the present disclosure.

FIG. 3 is a schematic side view of an exemplary wound dressing material 300 according to the present disclosure.

FIG. 4 is a schematic side view of an exemplary wound dressing material 400 according to the present disclosure.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

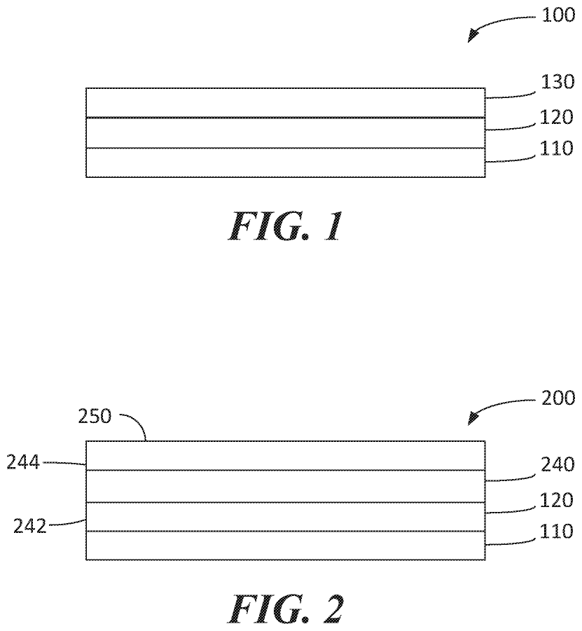

Wound dressing materials according to the present disclosure have various embodiments. Referring now to FIG. 1, wound dressing material 100 comprises first water-sensitive film 110 comprising a first copolymer comprising first divalent hydroxyethylene monomer units and first divalent dihydroxybutylene monomer units. Antimicrobial layer 120 is disposed on first water-sensitive film 110. Optional second water-sensitive film 130 is disposed on first antimicrobial layer 120 opposite the first water-sensitive film 110. In an embodiment, not shown here, optional second water-sensitive film 130 130 could be replaced by a water-sensitive nonwoven fabric, scrim, or mesh (e.g., made of the water-sensitive materials described herein).

Referring now to FIG. 2, wound dressing material 200 comprises first water-sensitive film 110, first antimicrobial layer 120 disposed on first water-sensitive film 110, and porous core layer 240 having first and second opposed major surfaces (242, 244). First major surface 242 of porous core layer 240 contacts first antimicrobial layer 120. Optional flexible adhesive barrier film 250 is adhered to and proximate to the second major surface 244 of porous core layer 240.

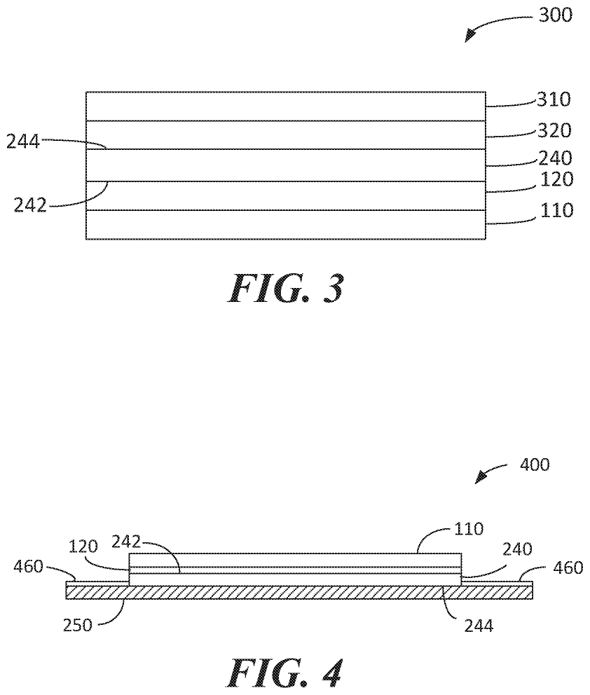

Referring now to FIG. 3, wound dressing material 300 comprises first water-sensitive film 110, first antimicrobial layer 120 disposed on first water-sensitive film 110, and porous core layer 240 having first and second opposed major surfaces (242, 244). First major surface 242 of porous core layer 240 contacts first antimicrobial layer 120. Second antimicrobial layer 320 is disposed on second major surface 244 of porous core layer 240. Second water-sensitive film 310 is disposed on second antimicrobial layer 320 opposite porous core layer 240.

The various components of the above-described wound dressing materials will now be discussed in greater detail.

The first and second water-sensitive films comprise respective first and optionally second copolymers (which may be the same or different). Each respective copolymer comprises divalent hydroxyethylene monomeric units

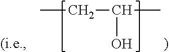

and divalent dihydroxybutylene monomer units. In some preferred embodiments, the divalent dihydroxybutylene monomer units comprise 3,4-dihydroxybutan-1,2-diyl monomer units

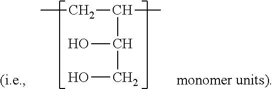

Optionally, but typically, the copolymer further comprises acetoxyethylene divalent monomeric units

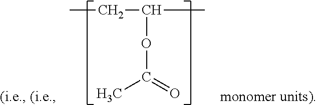

The copolymer may be obtained by copolymerization of vinyl acetate and 3,4-dihydroxy-1-butene followed by partial or complete saponification of the acetoxy groups to form hydroxyl groups.

Alternatively, in place of 3,4-dihydroxy-1-butene, a vinyl carbonate monomer such as

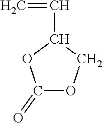

can also be used. After copolymerization, this carbonate may be hydrolyzed simultaneously with saponification of the acetate groups. In another embodiment, in place of 3,4-dihydroxy-1-butene, a vinyl acetal or ketal having the formula:

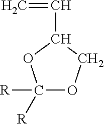

where each R is independently hydrogen or alkyl (e.g., methyl or ethyl) can be used. After copolymerization, this carbonate may be hydrolyzed simultaneously with saponification of the acetate groups, or separately. The copolymer can be made according to known methods or obtained from a commercial supplier, for example.

Commercially available copolymers may include those available under the trade designation Nichigo G-Polymer (Mitsubishi Chemical Company, Tokyo, Japan), a highly amorphous polyvinyl alcohol, that is believed to have divalent monomer units of hydroxyethylene, 3,4-dihydroxybutan-1,2-diyl, and optionally acetoxyethylene. Mitsubishi Chemical Company also refers to Nichigo G-Polymer by the chemical name butenediol vinyl alcohol (BVOH). Exemplary materials include Nichigo G-Polymer grades AZF8035W, OKS-1024, OKS-8041, OKS-8089, OKS-8118, OKS-6026, OKS-1011, OKS-8049, OKS-1028, OKS-1027, OKS-1109, OKS-1081, and OKS-1083. These copolymers are believed to have a saponification degree of 80 to 97.9 mole percent, and further contain an alkylene oxide adduct of a polyvalent alcohol containing 5 to 9 moles of an alkylene oxide per mole of the polyvalent alcohol. These materials have melt-processing properties that are suitable for forming fibers, nonwovens such as melt-blown and spunbond webs, and films.

The first and second water-sensitive films may optionally further contain (e.g., as a blend) bioabsorbable polymers in addition to the foregoing polymers. Useful bioabsorbable polymers may include, for example, fibers comprising: polycaprolactone; polylactide (PLA); polyglycolide (PGA); polydioxane; poly(glycolide-co-lactide) (PGA-co-PLA); poly(lactic acid-co-caprolactone); and copolyesters of ε-caprolactone, trimethylene carbonate, and p-dioxanone; and combinations thereof.

Preferably, bioabsorbable polymers used in wound dressing materials according to the present disclosure have a molecular weight (Mn) in the range of about 1,000 to about 8,000,000 g/mole, more preferably about 4,000 to about 250,000 g/mole, although this is not a requirement.

The water-sensitive film(s) may have any basis weight, but in many embodiments, it is preferably in the range of 50 to 1000 grams per square meter (gsm), more preferably 100 to 650 gsm, and more preferably 150 to 550 gsm. Likewise, the water-sensitive films may have any thickness, but typically are 100 microns to 1 millimeter in thickness, more typically 230 to 535 microns.

The water-sensitive film(s) may independently be perforated (i.e., having openings extending therethrough) or unperforated (i.e., continuous without openings therethrough). As used, herein the term “perforated” is not limited merely to opening formed by a punching or ablative process, but to openings formed by other processes (e.g., molding) as well. If perforated, the average diameter of the perforations may be 0.05 to 5 millimeters, preferably 0.1 to 0.5 millimeters, although perforations with other average diameters may be used as well. In some embodiments, at least some of the perforations can be slits, slots, circular, oval, square, and/or triangular in shape.

In some embodiments, useful water-sensitive films are uninterrupted and coextensive with the antimicrobial layers. In some embodiments, useful water-sensitive films can be provided as a series of discrete portions (e.g., strips) that collectively form a layer.

Antimicrobial layers (e.g., first and second antimicrobial layers) useful in practice of the present disclosure provide effective topical antimicrobial activity and thereby treat and/or prevent a wide variety of afflictions. For example, they can be used in the treatment and/or prevention of afflictions that are caused, or aggravated by, microorganisms (e.g., Gram positive bacteria, Gram negative bacteria, fungi, protozoa, mycoplasma, yeast, viruses, and even lipid-enveloped viruses) on skin. Particularly relevant organisms that cause or aggravate such afflictions include Staphylococcus spp., Streptococcus spp., Pseudomonas spp., Enterococcus spp., Acinetobacter spp., Klebsiella spp., Enterobacter spp., and Esherichia spp., bacteria, as well as herpes virus, Aspergillus spp., Fusarium spp., Candida spp., as well as combinations thereof. Particularly virulent organisms include Staphylococcus aureus (including resistant strains such as Methicillin Resistant Staphylococcus aureus (MRSA), Staphylococcus epidermidis, Group A and B Streptococcus, Streptococcus pneumoniae, Enterococcus faecalis, Vancomycin Resistant Enterococcus (VRE), Pseudomonas aeruginosa, Acinetobacter baumannii, Klebsiella pneumoniae, Escherichia coli, (including multidrug resistant (MDR) species thereof) Aspergillus niger, Aspergillus fumigatus, Aspergillus clavatus, Fusarium solani, Fusarium oxysporum, Fusarium chlamydosporum, Candida albicans, Candida glabrata, Candida krusei, Candida auris and combinations thereof.

In some embodiments, the antimicrobial layer(s) may be a surface coating (e.g., a paste or gel) on either or both of the first and second water-sensitive films, or it may be a freestanding layer (e.g., a film).

In some embodiments, the antimicrobial layer(s), when provided as a free thin film (i.e., not as a coating on a substrate) have a basis weight in the range of 20 to 700 gsm, more preferably in the range of 75 to 600 gsm, and more preferably in the range of 100 to 500 gsm, are typically flexible and can be deformed without breaking, shattering, or flaking of the antimicrobial layer.

Each antimicrobial layer comprises at least one antimicrobial compound. Exemplary antimicrobial compounds include antibiotics (e.g., amoxicillin, bacitracin zinc, doxycycline, cephalexin, ciprofloxacin, clindamycin, metronidazole, azithromycin, sulfamethoxazole, trimethoprim, or levofloxacin), and antiseptics such as chlorhexidine and its salts (e.g., chlorhexidine digluconate and chlorhexidine diacetate), antimicrobial lipids, phenolic antiseptics, cationic antiseptics, iodine and/or an iodophor, peroxide antiseptics, antimicrobial natural oils, alkane-1,2-diols having 6 to 12 carbon atoms, silver, silver salts and complexes, silver oxide, copper, copper salts, and combinations thereof. Preferred antimicrobial compounds include antimicrobial quaternary amine compounds (e.g., benzalkonium chloride) and salts thereof, cationic surfactants (e.g., cetylpyridinium chloride or cetyltrimethylammonium bromide), polycationic compounds such as octenidine or a salt thereof, biguanide compounds (e.g., chlorhexidine, polyhexamethylenebiguanide (PHMB) or a salt thereof, 1,2-organic diols having 6 to 12 carbon atoms (e.g., 1,2-octanediol), antimicrobial fatty acid monoester compounds, and combinations thereof.

Many preferred antimicrobial layers comprise an effective amount of a polycarboxylic acid chelator compound, alone or in combination with any of the foregoing antimicrobial compounds. The amount is effective to prevent growth of a microorganism and/or to kill microorganisms on a surface to which the composition is contacted.

In certain embodiments, the polycarboxylic acid chelator compound, whether aliphatic, aromatic, or a combination thereof, comprises at least two carboxylic acid groups. In certain embodiments, the polycarboxylic acid chelator compound, whether aliphatic, aromatic or a combination thereof, comprises at least three carboxylic acid groups. In certain embodiments, the polycarboxylic acid chelator compound, whether aliphatic or aromatic, comprises at least four carboxylic acid groups.

Polycarboxylic acid-containing chelator compounds suitable for use in the antimicrobial layer include aliphatic polycarboxylic acids, aromatic polycarboxylic acids, compounds with both one or more aliphatic carboxylic acids and one or more aromatic carboxylic acids, salts thereof, and combinations of the foregoing. Nonlimiting examples of suitable polycarboxylic acid-containing chelator compounds include citric acid, glutaric acid, glutamic acid, maleic acid, succinic acid, tartaric acid, malic acid, ethylenediaminetetraacetic acid, phthalic acid, trimesic acid, and pyromellitic acid.

Preferred salts include those formed from monovalent inorganic bases and include cations such as K⁺, Na⁺, Li⁺, and Ag⁺, and combinations thereof. In some compositions polyvalent bases may be appropriate and include cations such as Ca²⁺, Mg²⁺, and/or Zn²⁺. Alternatively, the salt of the polycarboxylic acid may be formed using an organic base such as a primary, secondary, tertiary, or quaternary amine.

In many embodiments, the polycarboxylic acid-comprising chelator compound may be present in the antimicrobial layer at relatively high concentrations (on a weight basis) while the composition remains surprisingly non-frangible. The minimum effective amount of chelator compound in the antimicrobial layer is related to the number of carboxyl groups in the chelator compound. For example, succinic acid (with two carboxyl groups) is generally more efficacious than glutamic acid having the same number of carboxylic acid groups since in glutamic acid carboxyl group forms a zwitterion with an amino group.

Mucic acid is another example with two carboxyl groups. Mucic acid is not as efficacious as succinic acid since the carboxyl groups are further apart and sterically hindered. In certain embodiments, efficacy of the composition can be improved by using thicker (greater basis weight) antimicrobial layers. Efficacy may depend on the amount of acid in the antimicrobial layer as well as the total amount (mass) of the antimicrobial layer. Thus, in some embodiments, the chelator compound comprises at least about 5, 10, 15, 20, 25, 30, 35, 40, 45, 50, 55, or even at least 60 percent by weight of an essentially solvent-free antimicrobial layer. The term “essentially solvent-free” is understood to mean that the antimicrobial layer has been processed to remove most of the solvent (e.g., water and/or organic solvent) or has been processed in such a way that no solvent (e.g., water and/or organic solvent) was required. Generally, solvents are relatively volatile compounds having a boiling point at one atmosphere pressure of less than 150° C. Solvent may be used to process (e.g., coat or film-form) the antimicrobial layer, but is preferably substantially removed to produce the final article for sale. For example, certain precursor compositions used to form the antimicrobial layer are first combined with water as a vehicle to form a solution, emulsion, or dispersion. These precursor compositions are coated and dried on a substrate (e.g., a disposable liner or the water-sensitive film(s)) such that the water content of the antimicrobial layer is less than 10 percent by weight, preferably less than 5 percent by weight, and more preferably less than 2 percent by weight.

In some embodiments, the chelator compound comprises up to about 15, 20, 25, 30, 35, 40, 45, 50, 55, or even up to about 60 percent by weight of the essentially dry antimicrobial layer on a weight basis.

In certain embodiments, wherein the polycarboxylic acid-comprising chelator compound comprises two aliphatic carboxylic acid groups (e.g., succinic acid), the chelator compound comprises at least about 10 percent by weight of the essentially dry antimicrobial layer on a weight basis. In certain embodiments, wherein the polycarboxylic acid-comprising chelator compound comprises three aliphatic carboxylic acid groups (e.g., citric acid), the chelator compound comprises at least about 10 percent by weight of the essentially dry antimicrobial layer on a weight basis. In certain embodiments, wherein the polycarboxylic acid-comprising chelator compound comprises four aliphatic carboxylic acid groups (e.g., ethylenediaminetetraacetic acid), the chelator compound comprises at least about 5 percent by weight of the essentially dry antimicrobial layer on a weight basis.

When preparing antimicrobial layers the polycarboxylic acid-containing chelator compound may be dissolved and/or dispersed in a water-soluble plasticizer component and optionally a solvent such as water. The plasticizer component has a boiling point greater than 105° C. and has a molecular weight of less than 5000 daltons. Preferably, the plasticizer component is a liquid at 23° C. Typically, but not necessarily, the plasticizer component is the most abundant solvent in the antimicrobial layer in which the polycarboxylic acid-containing chelator compound is dissolved and/or dispersed. In certain embodiments, wherein water is used to prepare the antimicrobial layer, substantially all of the water is subsequently removed (e.g., after the antimicrobial layer has been coated onto a substrate and heated to remove water).

In certain embodiments, the chelator compound comprises an aliphatic and/or aromatic polycarboxylic acid, in which two or more of the carboxylic groups are available for chelation without any zwitterionic interaction. Although potential zwitterionic interactions (e.g., such as in L-glutamic acid) may decrease antimicrobial efficacy relative to similar compounds (e.g., glutaric acid, succinic acid) that do not comprise α-amino groups, such zwitterionic compounds also exhibit antimicrobial activity. In addition, two or more carboxylic acid groups in the polycarboxylic acid-containing chelator compounds should be disposed in the chelator compound in sufficient proximity to each other or the compound should be capable of folding/conforming to bring the carboxylic acids sufficiently close to facilitate chelation of metal ions. In certain embodiments, the chelator compound comprises an aliphatic polycarboxylic acid or a salt thereof, an aromatic polycarboxylic acid or a salt thereof, or a combination thereof. In certain embodiments, the chelator compound comprises an aliphatic portion. In certain embodiments, the chelator compound comprises an aliphatic portion. The carboxylic acids may be disposed on the aliphatic portion and/or on the aromatic portion. Nonlimiting examples of chelator compounds that comprise an aliphatic portion with a carboxylic acid group disposed thereon and an aromatic portion with a carboxylic acid group disposed therein include 3-(2-carboxyphenyl)propionic acid, 3-(4-carboxyphenyl)propionic acid, and 4-[(2-carboxyphenyl)amino]benzoic acid.

In certain embodiments, efficacy of the antimicrobial layer(s) can be improved by depositing a higher amount of dried antimicrobial layer. Efficacy is dependent on concentration of chelator compound in the antimicrobial layer as well as total amount of the antimicrobial layer.

The antimicrobial layer may contain plasticizer, preferably bioabsorbable. Suitable plasticizers may include, for example, glycerol, a polyglycerol having 2-20 glycerin units, polyglycerols partially esterified with C₁-C₁₈ alkylcarboxylic acids having at least two free hydroxyl groups (e.g., hexaglycerol monolaurate, decaglycerol monolaurate, polyglyceryl-6 caprate, polyglyceryl-4 oleate, polyglyceryl-10 trilaurate and the like), polyethylene oxide, polyethylene glycol, polyethylene glycols initiated by any of the glycols discussed herein such as polyethylene glycol glyceryl ether, propylene glycol, dipropylene glycol, tripropylene glycol, 2-methyl-1,3-propanediol, sorbitol, dimethylisosorbide, pentaerythritol, trimethylolpropane, ditrimethylolpropane, a random ethylene oxide/propylene oxide (EO/PO) copolymer or oligomer, a block EO/PO copolymer or oligomer, and combinations thereof.

Plasticizer may be present in the antimicrobial layer at relatively high concentrations (on a weight basis). In some embodiments, plasticizer comprises at least about 10 percent by weight of the antimicrobial layer. In some embodiments, plasticizer comprises at least about 10, 15, 20, 25, 30, 35, 40, 45, 50, 55, 60, 65, 70, or even at least 75 percent by weight of the antimicrobial layer. In certain embodiments, the plasticizer component can act as a humectant. Advantageously, this can maintain a moist environment in a wound to help promote healing of wound tissue.

Advantageously, the relatively high concentration of plasticizer and/or water-soluble or water-dispersible polymer in the antimicrobial layer can function as a controlled-release modulator that facilitates delivery of the antimicrobial(s) over an extended period of time. In some embodiments, the plasticizer component can also function as an antimicrobial component.

Antimicrobial layers according to the present disclosure are preferably solid at 25° C. In certain embodiments, the antimicrobial layer(s) may comprise a solvent having a normal boiling point of less than or equal to 100° C. Nonlimiting examples of such solvents include water and lower (C₂-C₅) alcohols. Preferably, before use, the antimicrobial layer(s) comprises very little solvent (e.g., less than or equal to about 10 percent by weight) having a normal boiling point of less than or equal to 100° C. In some embodiments, the antimicrobial layer comprises less than 5 percent by weight, less than 4 percent by weight, less than 3 percent by weight, less than 2 percent by weight, or even less than 1 percent by weight (by weight) of a solvent having a normal boiling point of less than or equal to 100° C. In certain embodiments, the antimicrobial layer may be substantially free (before use) of such solvents or any compounds having a normal boiling point of less than 100° C.

In many preferred embodiments, the antimicrobial layer(s) comprise a water-soluble or water-dispersible polymer as a binder. The water-soluble or water-dispersible polymer has a Tg greater than or equal to 20° C. In use, the polymer can function to form the antimicrobial layer into a cohesive shape such as a film while also absorbing wound exudate and to maintain a moist environment that can facilitate healing of the tissue at a wound site.

Exemplary water-soluble and/or water-dispersible polymers that are suitable for use in a antimicrobial layer according to the present disclosure include polyvinylpyrrolidone; polyvinyl alcohol; copolymers of vinyl alcohol; polybutylenediol; polysaccharides (e.g., starch); guar gum; locust bean gum; carrageenan; hyaluronic acid; agar; alginate; tragacanth; gum arabic; gum karraya; gellan; xanthan gum; hydroxyethylated, hydroxypropylated, and/or cationic derivatives of the foregoing; modified cellulose polymers (e.g., hydroxyethylcellulose, hydroxypropyl methylcellulose, carboxymethylcellulose, or cationic cellulose such as polyquaterium 4); copolymers of polyvinylpyrrolidone and vinyl acetate; water-soluble and water-swellable polyacrylates (e.g., based on hydroxyethyl (meth)acrylate, hydroxypropyl (meth)acrylate, (meth)acrylic acid, (meth)acrylamide, PEG (meth)acrylates, methyl (meth)acrylate), and combinations thereof. As used herein the term “(meth)acryl” refers to acryl and/or methacryl. In certain embodiments, the water-soluble or water-dispersible polymers can comprise a polyquaternium polymer.

In some embodiments, the water-soluble or water-dispersible polymer comprises at least about 5 percent by weight of the antimicrobial layer. In some embodiments, the water-soluble or water-dispersible polymer comprises up to about 65 percent by weight of the antimicrobial layer.

A variety of other ingredients may be added to the antimicrobial layers according to the present disclosure for desired effect. These include, but are not limited to, surfactants, skin emollients and humectants such as, for example, those described in U.S. Pat. No. 5,951,993 (Scholz et al.), fragrances, colorants, and/or tackifiers.

Some embodiments of wound dressing materials according to the present disclosure include a porous core layer. Useful porous core layers are preferably self-supporting, although this is not a requirement. The porous core layer may comprise a nonwoven fiber web, a porous foam, or a combination thereof, for example. The porous core layer is preferably sufficiently porous that wound exudate can pass through them in use. The porous core layer is preferably at least sufficiently flexible to easily conform to anatomical features associated with wounds, although this is not a requirement.

Useful nonwoven fiber web core layers comprise base fibers. The base fibers may be staple, and/or continuous. For example, the nonwoven fiber web core layers may comprise an entangled staple fiber web, a meltblown fiber web, or a spunbond fiber web. Staple fibers may be entangled by needle tacking and/or hydroentanglement, for example. The nonwoven fiber web core layers fibers may have any average diameter and/or length, preferably from 2 to 200 microns and more preferably 2 to 100 microns.

While nonwoven fiber web core layers may have any basis weight, in many embodiments, it is preferably in the range of 20 to 500 grams per square meter (gsm), more preferably 50 to 400 gsm, and more preferably 75 to 300 gsm.

In some embodiments, for example, where resistance to exudate and/or water swellability/solubility is desired, the base fibers may comprise polyolefin(s) (e.g., polyethylene (HDPE, LDPE, LLDPE, VLDPE; ULDPE, UHMW-PE), polypropylene, polybutylene, poly(1-butene), polyisobutylene, poly(1-pentene), poly(4-methylpent-1-ene), polybutadiene, or polyisoprene), polyester(s) (e.g., polylactic acid, polybutylene terephthalate, and polyethylene terephthalate), polyvinyl chloride, polymethyl methacrylate, polyacrylonitrile and copolymer(s) of acrylonitrile, polyamide(s) (e.g., polycaprolactam or nylon 6,6), polystyrene(s), polyphenylene sulfide(s), polysulfone(s), polyoxymethylene(s), polyimide(s), polyurea(s), hydrophobic thermoplastic polyurethane(s), styrenic block copolymer(s) (e.g., styrene-isoprene-styrene (SIS) block copolymers, styrene-ethylene-butadiene-styrene (SEBS) block copolymers, or styrene-butadiene-styrene (SBS) block copolymers), metal (e.g., stainless steel, nickel, tin, silver, copper, or aluminum fibers), glass fibers, ceramic fibers, natural fiber(s) (e.g., cotton fibers, wool fibers, cashmere fibers, kenaf fibers, jute fibers, flax fibers, hemp fibers, cellulosic fibers, sisal fibers, coir fibers), or any combination thereof.

In some embodiments, for example, where solubility and/or swellability in exudate and/or water is desired, the base fibers may comprise polyvinyl alcohol(s), carboxymethyl cellulose, rayon, cotton, cellulose acetate, hydrophilic thermoplastic polyurethane(s), chitosan, polyacrylic acid, sulfonated cellulose, cellulose ethyl sulfonate, alginate, or any combination thereof.

Blends of fibers with and without resistance to exudate and/or water swellability/solubility can also be used.

Useful porous foam core layers can be a porous polymeric foam, although this is not a requirement. The porous foam core can be crosslinked or uncrosslinked. Useful porous foams are preferably open-cell foams, although perforated or slit closed cell-foams may also be useful, for example,

Preferably, porous foam core layers are absorbent, and more preferably comprise an absorbent, substantially non-swellable foam. In this context, an “absorbent” foam is one that is capable of absorbing saline water, and hence, exudate from a wound. Preferably, suitable porous foam core layers are those that can absorb greater than 250%, more preferably at least about 500%, and most preferably at least about 800%, by weight aqueous saline solution based on the dry weight of the porous foam core layer. Typically, these values are obtained using a saline absorbency test in which a dry, weighed sample is immersed for 30 minutes at 37° C. in phosphate-buffered saline containing 0.9 wt. % NaCl.

The porous foam core layers can be of a wide range of thicknesses. Preferably, they are at least about 0.5 millimeter, and more preferably at least about 1 millimeter thick. Preferably, they are no greater than about 80 millimeters, and more preferably no greater than about 30 millimeters thick.

Furthermore, they can include one or more layers tailored to have the desired properties. These layers can be directly bonded to each other or bonded together with adhesive layers, for example, as long as the overall properties of the wound dressing, as described herein, are met. Optionally, disposed between these layers can be one or more layers of polymeric netting or nonwoven, woven, or knit webs for enhancing the physical integrity of the porous foam core layer. Suitable open cell foams preferably have an average cell size (typically, the longest dimension of a cell, such as the diameter) of at least about 30 microns, more preferably at least about 50 microns, and preferably no greater than about 800 microns, more preferably no greater than about 500 microns, as measured by scanning electron microscopy (SEM) or light microscopy. Such porous foam core layers when used in dressings of the present disclosure allow transport of fluid and cellular debris into and within the foam. Preferably, the porous foam core layer includes a synthetic polymer that is adapted to form a conformable open cell foam that absorbs the wound exudate.

Exemplary porous foam core layers can comprise porous foams made of polyolefin (e.g., polyethylene or polypropylene), polystyrene, polyurethane, polyacrylate, polyester (e.g., polylactic acid), polycarbonate, polyamide, carboxylated butadiene-styrene rubbers, and combinations thereof. These materials are commercially available and/or can be made by known extrusion methods such as polymer (gas) saturation or inclusion of a foaming agent. Polyurethane foams are often preferred.

A particularly preferred foam is a polyurethane, available under the trade designation POLYCRIL 400 from Fulflex, Inc, Middleton, Rhode Island. Although suitable foams may be hydrophilic per se, they are preferably hydrophobic and treated to render them more hydrophilic, for example with surfactants such as nonionic surfactants, e.g., the oxypropylene-oxyethylene block copolymers available under the trade designation PLURONIC from BASF Wyandotte, Mount Olive, New Jersey. Use of foams, or surfactants incorporated therein, that possess a hydrophilic surface reduces the tendency for the exudate to coagulate rapidly in the foam. This helps to keep the wound in a moist condition even when production of exudate has ceased from the wound.

Further details concerning suitable polymer foams can be found in U.S. Pat. No. 6,548,727 (Swenson), the disclosure of which is incorporated herein by reference.

Some embodiments of wound dressing materials according to the present disclosure include an adhesive barrier film. Commercially available suitable flexible adhesive barrier films are marketed by 3M Company under the trade designation TEGADERM (e.g., 3M TEGADERM Transparent Film Roll), by Johnson & Johnson Company, New Brunswick, New Jersey under the trade designation BIOCLUSIVE, and by T. J. Smith & Nephew, Hull, England under the trade designation OP-SITE.

Wound dressing materials can be made, for example, using known methods of assembling laminated/layered structures in which the various component layers may be laminated (e.g., using pressure and/or heat) in the desired order. Alternatively, individual layers (e.g., water-sensitive layers, antimicrobial layers) may be coated out of water and/or organic solvent and at least partially dried instead of lamination. Coating methods may include, for example, roll coating, spray coating, stencil-printing, screen-printing, flexography, knife coating, and slot coating.

In the case of adhesive barrier films, they may be advantageously releasably adhered to a disposable liner in some cases; for example, if the adhesive film extends beyond the other layers/films of the wound dressing material.

Referring now to FIG. 4, exemplary wound dressing material 400 comprises porous core layer 240. Antimicrobial layer 120 is sandwiched between first major surface 242 of porous core layer 240 and water-sensitive film 110. Flexible adhesive barrier film 250 is adhered to and proximate to second major surface 244 of porous core layer 240. In the particular embodiment shown, flexible adhesive barrier film 250 extends beyond the periphery of the other components such as water-sensitive film 110, antimicrobial layer 120, and porous core layer 240, so that it may stick to the skin surrounding the wound, however this is not a requirement. In this embodiment, the exposed adhesive side of the adhesive barrier film may be protected by a disposable protective releasable liner 460.

Wound dressing materials according to the present disclosure may have broad-spectrum antimicrobial activity. However, the wound dressing materials are typically sterilized; for example, by sterilized by a variety of industry standard techniques. For example, it may be preferred to sterilize the wound dressing materials in their final packaged form using electron beam. It may also be possible to sterilize the sample by gamma radiation, nitrogen dioxide sterilization and/or heat. Other forms of sterilization may also be used. It may also be suitable to include preservatives in the formulation to prevent growth of certain organisms. Suitable preservatives include industry standard compounds such as parabens (e.g., methylparaben, ethylparaben, propylparaben, isopropylparaben, or isobutylparaben); 2 bromo-2 nitro-1,3-diol; 5 bromo-5-nitro-1,3-dioxane, chlorbutanol, diazolidinyl urea; iodopropyl butyl carbamate, phenoxyethanol, halogenated cresols, methylchloroisothiazolinone; and combinations thereof.

Wound dressing materials according to the present disclosure are useful, for example, for placement in a wound. Typically, the exposed surface of the wound is cleaned and/or treated with antiseptic (if necessary) and then contacted with the wound dressing material, which may be placed within the wound to facilitate healing.

Wound dressing materials according to the present disclosure are normally designed to be used by contacting the first water-sensitive film with an exposed surface of a wound. In embodiments with an adhesive barrier film, the wound dressing material is contacted with an exposed surface of a wound, and the adhesive barrier film is adhered to skin adjacent to at least a portion of the wound. In some of these embodiments, the wound is closed over by the wound dressing material which then provides antiseptic agents to facilitate healing, and ultimately being absorbed by the body.

When contacted with a wound site, some or all of the wound dressing material of the present disclosure is hydrated by the tissue fluids and wound exudate.

Wound dressing materials according to the present disclosure may have any basis weight, thickness, porosity, and/or density unless otherwise specified. Wound dressing materials according to the present disclosure may have any desired thickness. In many embodiments, the basis weight is in the range of 20 to 800 gsm, more preferably 60 to 600 gsm and more preferably 100 to 500 gsm.

The wound dressing material may be provided in roll form, or it may be converted into sheets or bandages (optionally further comprising a peripheral supporting frame).

Preferably, to maintain a low relative humidity, the wound dressing material should be packaged in a package with a low moisture vapor transmission rate (MVTR) such as, for example, a Techni-Pouch package (Technipaq, Inc., Crystal Lake, Illinois) with a PET/Aluminum Foil/LLDPE material construction.

Objects and advantages of this disclosure are further illustrated by the following non-limiting examples, but the particular materials and amounts thereof recited in these examples, as well as other conditions and details, should not be construed to unduly limit this disclosure.

### EXAMPLES

Unless otherwise noted, all parts, percentages, ratios, etc. in the Examples and the rest of the specification are by weight.

### Preparation of Film Layers

Polymer butanediol vinyl alcohol copolymer (BVOH) (obtained as Nichigo G-Polymer OKS 8112 pellets from the Mitsubishi Chemical Corporation, Tokyo, Japan). blends were made at different blend ratios of BVOH with two different molecular weights of polyethylene glycerol (PEG), 200 g/mol (PEG200) or 400 gm/mol (PEG400) both obtained from Spectrum Chemical Company, New Brunswick NJ. Each sample was melt-blended in a twin screw microcompounder manufactured by Xplore Instruments, Sittard, The Netherlands. A processing temperature of 205° C. was maintained by the machine's six individual heating zones, the screw speed was set at 100 rpm, and a mixing force of 1 kilonewton was used. The polymer mixture was pushed into the feed port and allowed to circulate in the compounder for five minutes before being removed. After removal, the sample was placed between two smooth metal plates and made into a film using a hydraulic press. Two different shims were used between the metal plates to generate films of two different thicknesses. Some films we then perforated using a calendar with a steel roll and a sawtooth pattern roll. The rolls were heated to 250° F. (121° C.) and run at a speed of 4 feet/min (1.2 m/min) with a force of 1200 lb/in (2.1 kN/cm). The film samples are described in Table 1, below.

TABLE 1

| Film Layer | | | | Thickness, | | |
|---|---|---|---|---|---|---|
| Sample | | | | mils | Basis Weight | Perforated |
| Number | % BVOH | % PEG2000 | % PEG4000 | (microns) | g/m² | or not |
| 1 | 100 | 0 | 0 | 10.9 (277) | 336 | perforated |
| 2 | 100 | 0 | 0 | 30.5 (775) | 957 | not |
| | | | | | | perforated |
| 5 | 90 | 10 | 0 | 18.1 (460) | 528 | not |
| | | | | | | perforated |
| 10 | 90 | 10 | 0 | 11.5 (292) | 354 | perforated |
| 11 | 80 | 20 | 0 | 9.9 (251) | 302 | not |
| | | | | | | perforated |
| 12 | 80 | 20 | 0 | 10.9 (277) | 337 | perforated |
| 27 | 80 | 20 | 0 | 21.8 (554) | 607 | perforated |
| 33 | 70 | 30 | 0 | 9.8 (249) | 368 | not |
| | | | | | | perforated |
| 17 | 70 | 30 | 0 | 9.2 (234) | 308 | perforated |
| 34 | 90 | 0 | 10 | 11.4 (290) | 368 | not |
| | | | | | | perforated |
| 35 | 90 | 0 | 10 | 8.1 (206) | 336 | perforated |
| 19 | 80 | 0 | 20 | 12.5 (318) | 383 | not |
| | | | | | | perforated |
| 49 | 80 | 0 | 20 | 8.65 (220) | 361 | perforated |
| 50 | 80 | 0 | 20 | 9.7(246) | 422 | not |
| | | | | | | perforated |
| 51 | 70 | 0 | 30 | 10.65 (271) | 381 | perforated |
| 53 | 70 | 0 | 30 | 11.25 (296) | 411 | not |
| | | | | | | perforated |

### Preparation of Antimicrobial Composition for the Antimicrobial Layer

An antimicrobial composition was prepared in a 100 g batch using the components listed in Table 2. All components except the L-PVPK60 were added to a MAX 100 mixing cup (Flacktec Incorporated, Landrum, SC) and mixed at 3500 rpm (revolutions per minute) for 1 minute using a DAC 400 FVZ SPEEDMIXER instrument (Flacktec). The L-PVPK60 aqueous mixture was added to the cup and the contents were mixed for 1 minute at 3500 rpm.

The viscous composition was knife-coated onto a release liner using a gap of 254 micrometers. The coating was then dried at 80° C. for 10 minutes in a convection oven to produce a coating with a basis weight of 100 g/m².

TABLE 2

| | WEIGHT | |
|---|---|---|
| COMPONENT | PERCENT | SOURCE |
| Glycerol | 19 | Cargill Corporation, |
| | | Wayzata, Minnesota |
| Linear polyvinylpyrrolidone | 50 | Ashland Incorporated, |
| K60, 47% in water | | Covington, Kentucky |
| (L-PVPK60) | | |
| Benzalkonium chloride 50% | 0.3 | Novo Nordisk Pharmatech, |
| (BAC) | | Koge, Denmark |
| Capryl glycol (Hydrolite 8) | 0.6 | Symrise AG, Holzminden, |
| | | Germany |
| Sterile water | 12.6 | Rocky Mountain Biologicals, |
| | | Missoula, Montana |
| Sodium Citrate | 10 | MilliporeSigma, |
| | | St. Louis, Missouri |
| Citric acid monohydrate | 7.5 | MilliporeSigma |

### Preparation of a Base Fiber Layer

A multicomponent BMF web was made using a melt blowing process similar to that described in V. A. Wente, “Superfine Thermoplastic Fibers” in Industrial Engineering Chemistry, Vol. 48, pages 1342 et seq. (1956). The extruder feeding molten (co)polymer to the melt-blowing die was a STEER 20-mm twin screw extruder commercially available from STEER Co., equipped with two weight loss feeders to control the feeding of the (co)polymer resins to the extruder barrel and a melt pump to control the (co)polymer melt flow to a melt-blowing die. The die had a plurality of circular smooth surfaced orifices (10 orifices/cm) with a 5.1 diameter ratio as generally described in are described in, e.g., U.S. Pat. No. 5,232,770 (Joseph et al.).

The web example discussed below were made using an apparatus equipped with a multi-layer feed block configured to obtain multi-component blown micro-fibers that exhibit an axial cross-sectional structure, when the fiber is viewed in axial cross-section, consisting of three layers.

The BMF web was made with each fiber having 3 layers. The inner layer of the fiber was made of Tecophilic TPU TG2000 (obtained from Lubrizol) and the outer layers were made using a Dow DNDA 1081 Linear low-density polyethylene (LLDPE) from the Dow Chemical Company, Midland, Michigan.

The two extruders were kept at the same temperature at 210° C. to deliver the melt stream to the BMF die (maintained at 210° C.). The gear pumps were adjusted to obtain a 75/25 ratio of Tecophilic TPU TG2000/LLDPE with a total polymer throughput rate of 0.178 kg/hr/cm die width (1.0 lb/hr/inch die width) maintained at the BMF die. The primary air temperature was maintained at approximately 325° C. The BMF webs were directed to the drum collector, in between the BMF die and drum collector, PET staple fibers were dispensed into the melt-blown fibers. The crimped staple PET fibers were 3.3 dtex and length of 38.1 mm and was obtained from Invista, Wichita, Kansas. Enough staple fibers were dispensed so as to constitute 30% by weight of the final nonwoven web.

The resulting web was collected at a BMF die to collector distance of 21 inches and a collection rate of 7.1 ft/min. The resultant nonwoven fiber web had a nominal basis weight of 207 g/m². The resulting fibers had fiber diameter in the range of 5 to 30 micrometers.

### Example 1

An antimicrobial wound care article with a layered construction was assembled. A 4 in×4 in (10.2 cm×10.2 cm) BVOH film (film 2) was laminated to each side of the antimicrobial layer using had pressure to form the article.

### Example 2

An antimicrobial wound care article with a layered construction was assembled by first laminating an antimicrobial layer to one surface of the core fiber core material and then peeling off the release liner. Then, a 2 in×4 in (5.1 cm×10.2 cm) piece of the BVOH film (film 5) was laminated to one side using hand pressure. Finally, the entire construction was trimmed so that the final construction was 2 in×4 in (5.1 cm×10.2 cm).

### Example 3

A 2 in×4 in (5.1 cm×10.2 cm) piece of the antimicrobial layer was laminated to the exposed side of the core fiber material from example 2 using hand pressure and then peeling off the release liner. Then, a 2 in×4 in (5.1 cm×10.2 cm) piece of BVOH film 5 was laminated to that antimicrobial layer using hand pressure to create the final article.

### Example 4

An antimicrobial wound care article was assembled in an identical manner as the article in Example 3, except the BVOH film was film sample number 19.

### Example 5

An antimicrobial wound care article was assembled in an identical manner as the article in Example 4, except the core layer was SAQ hydrophilic polyurethane foam, standard grade, natural color, 0.5 cm thickness (Crest Foam Industries, Moonachie, New Jersey) and the BVOH film was film sample number 33.

### Example 6

An antimicrobial wound care article was assembled in an identical manner as the article in Example 1, except the BVOH film was film sample 1.

### Example 7

An antimicrobial wound care article was assembled in an identical manner as the article in Example 3, except the BVOH film was film sample 10.

### Example 8

An antimicrobial wound care article was assembled in an identical manner as the article in Example 3, except the BVOH film was film sample 49.

### Example 9

An antimicrobial wound care article was assembled in an identical manner as the article in Example 5, except the core layer was SAQ hydrophilic polyurethane foam, standard grade, natural color, 0.5 cm thickness (Crest Foam Industries), and the BVOH film was film sample 17.

### Example 10

A finished wound dressing material from Example 2 was placed on a hard flat surface with the scrim layer of the construction facing the surface. A 6 in by 6 in (15.2 cm×15.2 cm) square section of transparent barrier film was cut from a roll of 6 in (15.2 cm) wide 3M TEGADERM transparent barrier film (obtained from 3M Company) and the backing layer was removed to expose the adhesive surface of the barrier film. The barrier film was centered with respect to the wound dressing material and adhesively adhered to base fiber web surface of the wound dressing material (i.e., adhesive surface of the TEGADERM barrier film in contact with the base fiber web). In this construction, the barrier film extended beyond the outer edges of the scrim and the base fiber web.

All cited references, patents, and patent applications in this application that are incorporated by reference, are incorporated in a consistent manner. In the event of inconsistencies or contradictions between portions of the incorporated references and this application, the information in this application shall control. The preceding description, given in order to enable one of ordinary skill in the art to practice the claimed disclosure, is not to be construed as limiting the scope of the disclosure, which is defined by the claims and all equivalents thereto.

1. A wound dressing material comprising:
a first water-sensitive film comprising a first copolymer comprising first divalent hydroxyethylene monomer units and first divalent dihydroxybutylene monomer units; and
a first antimicrobial layer disposed on the first water-sensitive film, wherein the first antimicrobial layer comprises a polycarboxylic acid-comprising a chelator compound that comprises two aliphatic carboxylic acid groups, and wherein the chelator compound comprises at least about 10 percent by weight of the essentially dry antimicrobial layer on a weight basis.

2. The wound dressing material of claim 1, further comprising a second water-sensitive film comprising a second copolymer comprising second divalent hydroxyethylene monomer units and second divalent dihydroxybutylene monomer units, wherein the second water-sensitive film is disposed on the first antimicrobial layer opposite the first water-sensitive film.

3. The wound dressing material of claim 2, wherein the second copolymer further comprises second divalent acetoxyethylene monomer units.

4. The wound dressing material of claim 2, wherein the second divalent dihydroxybutylene monomer units comprise second divalent 3,4-dihydroxybutan-1,2-diyl monomer units.

5. The wound dressing material of claim 1, further comprising a porous core layer having first and second opposed major surfaces, wherein the first major surface of the porous core layer contacts the first antimicrobial layer.

6. The wound dressing material of claim 5, wherein the porous core layer comprises a foam.

7. The wound dressing material of claim 5, wherein the porous core layer comprises a nonwoven web.

8. The wound dressing material of claim 7, further comprising a flexible adhesive barrier film adhered to and proximate to the second major surface of the porous core layer.

9. The wound dressing material of claim 8, further comprising:
a second antimicrobial layer disposed on the second major surface of the porous core layer; and
a second water-sensitive film disposed on the second antimicrobial layer opposite the porous core layer, wherein the second water-sensitive film comprises a second copolymer comprising divalent hydroxyethylene monomer units and divalent dihydroxybutylene monomer units.

10. The wound dressing material of claim 9, wherein the second copolymer further comprises second divalent acetoxyethylene monomer units.

11. The wound dressing material of claim 9, wherein the second divalent dihydroxybutylene monomer units comprise second divalent 3,4-dihydroxybutan-1,2-diyl monomer units.

12. The wound dressing material of claim 1, wherein the first copolymer further comprises first divalent acetoxyethylene monomer units.

13. The wound dressing material of claim 1, wherein the first divalent dihydroxybutylene monomer units comprise first divalent 3,4-dihydroxybutan-1,2-diyl monomer units.

14. The wound dressing material of claim 1, wherein the first water-sensitive film is perforated.

15. The wound dressing material of claim 1, wherein the first water-sensitive film is uninterrupted.

16. The wound dressing material of claim 2, wherein the second water-sensitive film is perforated.

17. The wound dressing material of claim 2, wherein the second water-sensitive film is uninterrupted.

18. A method of using the wound dressing material of claim 1, the method comprising contacting the first water-sensitive film with an exposed surface of a wound.

19. A method of using the wound dressing material of claim 8, the method comprising contacting the first water-sensitive film of the wound dressing material with an exposed surface of a wound, and adhering the flexible adhesive barrier film to skin adjacent to at least a portion of the wound.

20. A method of making a wound dressing material, the method comprising laminating sequential layers:
a) a first water-sensitive film comprising a first copolymer comprising first divalent hydroxyethylene monomer units and first divalent dihydroxybutylene monomer units; and
b) a first antimicrobial layer, wherein the first antimicrobial layer comprises a polycarboxylic acid-comprising a chelator compound that comprises two aliphatic carboxylic acid groups, and wherein the chelator compound comprises at least about 10 percent by weight of the essentially dry antimicrobial layer on a weight basis; and optionally
c) a porous core layer.

21. The method of claim 20, wherein the sequential layers further comprise:
d) a second antimicrobial layer; and
e) a second water-sensitive film comprising a second copolymer comprising second divalent hydroxyethylene monomer units and second divalent dihydroxybutylene monomer units.

22. The method of claim 20, wherein the sequential layers further comprise:
d) a flexible adhesive barrier film.
